(19) United States
(12) Patent Application Publication
(10) Pub. No.: US 2011/0184011 A1
(43) Pub. Date: Jul. 28, 2011

(54) Compounds

(75) Inventors: Andrew Jones, Essex (GB); Carol Austin, Essex (GB)

(73) Assignee: SELCIA LIMITED, Essex (GB)

(21) Appl. No.: 13/063,508

(22) PCT Filed: Sep. 11, 2009
(86) PCT No.: PCT/GB2009/051171

§ 371 Date: Apr. 1, 2011

(30) Foreign Application Priority Data
Sep. 12, 2008 (GB) 0816759.5

(51) Int. Cl. A61K 31/437; C07D 471/04; A61P 27/02; A61P 19/02; A61P 17/00

(52) U.S. Cl. 514/299; 546/112

(57) ABSTRACT

The present invention relates to new therapeutic and prophylactic applications of known compounds of general formula (Ia), novel compounds of formula (Ia)

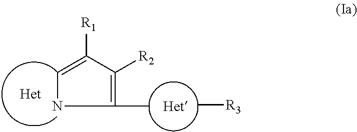

wherein R3 is methyl, and to compositions comprising them, useful as a medication for the topical therapeutic and prophylactic treatment of dermatological and ophthalmic disorders, rheumatoid arthritis and osteoarthritis.

### CROSS-REFERENCE TO RELATED APPLICATIONS

The present application is a National Stage Application claiming the priority of co-pending PCT Application No. PCT/GB2009/051171 filed Sep. 11, 2009, which in turn, claims priority from Great Britain Application Serial No. GB0816759.5, filed Sep. 12, 2008. Applicants claim the benefits of 35 U.S.C. 120 as to the PCT application and priority under 35 U.S.C. §119 as to the said Great Britain application, and the entire disclosures of both applications are incorporated herein by reference in their entireties.

The present invention relates to new therapeutic and prophylactic topical applications of known compounds, novel compounds, and to compositions comprising them.

Compounds of general formula (I) and pharmaceutically acceptable salts thereof as described below are known from U.S. Pat. No. 6,194,426:

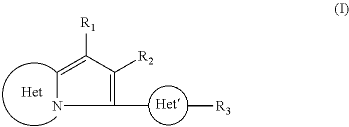

in which
Het denotes a ring condensed with pyrrole, such that it forms a pyrrolothiazole, 5,6,7,8-tetrahydroindolizine, dihydropyrrolothiazine or dihydropyrrolizine ring,
R1 is a carboxamide, cyano, carboxyl, alkoxy carbonyl, acyl or imidazolylcarbonyl radical,
R2 is a hydrogen or halogen atom or an alkyl, alkenyl, trihalomethyl or cyano radical,
R3 is a hydrogen or halogen atom or a hydroxyl or alkyl radical, and
Het′ is a pyridyl, pyridyl N-oxide or thiazolyl radical,
wherein the alkyl or acyl radicals are straight or branched and contain 1 to 4 carbon atoms and the alkenyl radicals are straight or branched and contain 2 to 4 carbon atoms, and
wherein, when R2 and/or R3 are halogen, they are chosen from chlorine, bromine, fluorine or iodine.

U.S. Pat. No. 6,207,675 covers compounds of similar formulae for the treatment and prevention of conditions in which viruses of the Herpes family are involved and/or in which cytokines, including TNF_{α} are involved.

It has now been found that the compounds of general formula (I) and related compounds, can be useful as a medication for the topical therapeutic and prophylactic treatment of dermatological and ophthalmic disorders, and more particularly of such disorders which are not viral or infectious diseases which involve tumour necrosis factor (TNF_{α}).

In a first aspect therefore, the invention provides a method of topical treatment of dermatological and ophthalmic disorders in mammals by administration of a compound of formula (Ia)

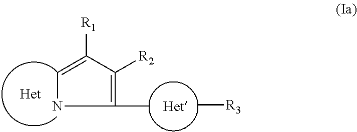

in which Het denotes a heterocyclic or carbocyclic ring condensed with a pyrrole such that it forms a substituted or unsubstituted indolizine, pyrrolothiazole, 5,6,7,8-tetrahydroindolizine, 5,6,-dihydroindolizine, dihydropyrrolothiazine or dihydropyrrolizine ring,
R1 is a carboxamide, cyano, carboxyl, alkoxy carbonyl or acyl,
R2 is a hydrogen or halogen atom or an alkyl, alkenyl, trihalomethyl or cyano radical,
R3 is a hydrogen or halogen atom or a hydroxyl or alkyl radical, and Het′ is a pyridyl, pyridyl N-oxide or thiazolyl radical,
wherein the alkyl or acyl radicals are straight or branched and contain 1 to 4 carbon atoms and the alkenyl radicals are straight or branched and contain 2 to 4 carbon atoms, and wherein, when R2 and/or R3 are halogen, they are chosen from chlorine, bromine, fluorine or iodine; or a pharmaceutically acceptable salt thereof.

Compounds in which Het denotes a heterocyclic or carbocyclic ring condensed with a pyrrole such that it forms a substituted indolizine, pyrrolothiazole, 5,6,7,8-tetrahydroindolizine, 5,6,-dihydroindolizine, dihydropyrrolothiazine or dihydropyrrolizine ring, may be substituted with for example hydrogen, alkyl, alkoxy, hydroxyl, cyano or halogen.

Moreover, Het′ may be further substituted in addition to the substituents of R3, by for example hydrogen, alkyl, alkoxy, hydroxyl, cyano or halogen.

Particular dermatological disorders for which compounds of formula Ia may used include atopic dermatitis, contact dermatitis, seborrhaeic dermatitis, dermatitis herpetiformis, eczema, erythema, granuloma annulare, and inflammation caused by nettles, insect bites, poison ivy and/or poison oak, psoriasis and psoriatic arthritis.

The invention also includes the compounds of formula (Ia) for use in the topical treatment of inflammatory disorders such as rheumatoid arthritis and osteoarthritis.

Particular ophthalmic disorders include conjunctivitis, allergic conjunctivitis, dry eye disease, uveitis, blepharitis, meibomianitis, keratitis, conjunctival hyperaemia, Graves' ophthalmopathy, and Sjogren's syndrome.

The invention further provides a method of topical treatment of disorders in mammals which do not involve TNF_{α} and are not viral or infectious diseases, in particular dermatological disorders including atopic dermatitis, contact dermatitis, seborrhaeic dermatitis, dermatitis herpetiformis, eczema, erythema, granuloma annulare, and inflammation caused by nettles, insect bites, poison ivy and/or poison oak, psoriasis and psoriatic arthritis and ophthalmic disorders including conjunctivitis, allergic conjunctivitis, dry eye disease, uveitis, blepharitis, meibomianitis, keratitis, conjunctival hyperaemia, Graves' ophthalmopathy, and Sjogren's syndrome, by administration of a compound of formula (Ia) or a pharmaceutically acceptable salt thereof.

The invention also provides the use of a compound of formula (Ia) or a pharmaceutically acceptable salt thereof in the manufacture of a medicament for the topical treatment of dermatological and ophthalmic disorders in mammals.

The invention provides the use of a compound of formula (Ia) or a pharmaceutically acceptable salt thereof in the manufacture of a medicament for the topical treatment of disorders in mammals which do not involve TNF_{α} and are not viral or infectious diseases, in particular dermatological disorders including atopic dermatitis, contact dermatitis, seborrhaeic dermatitis, dermatitis herpetiformis, eczema, erythema, granuloma annulare, and inflammation caused by nettles, insect bites, poison ivy and/or poison oak, psoriasis and psoriatic arthritis and ophthalmic disorders including conjunctivitis, allergic conjunctivitis, dry eye disease, uveitis, blepharitis, meibomianitis, keratitis, conjunctival hyperaemia, Graves' ophthalmopathy, and Sjogren's syndrome.

The invention also provides the use of a compound of formula (Ia) or a pharmaceutically acceptable salt thereof for the topical treatment of disorders in mammals which do not involve TNF_{α} and are not viral or infectious diseases, in particular dermatological disorders including atopic dermatitis, contact dermatitis, seborrhaeic dermatitis, dermatitis herpetiformis, eczema, erythema, granuloma annulare, and inflammation caused by nettles, insect bites, poison ivy and/or poison oak, psoriasis and psoriatic arthritis and ophthalmic disorders including conjunctivitis, allergic conjunctivitis, dry eye disease, uveitis, blepharitis, meibomianitis, keratitis, conjunctival hyperaemia, Graves' ophthalmopathy, and Sjogren's syndrome.

The invention further provides a compound of formula (Ia) or a pharmaceutically acceptable salt thereof for use in the treatment of dermatological and ophthalmic disorders in mammals which do not involve TNF_{α} and are not viral or infectious diseases, in particular dermatological disorders including atopic dermatitis, contact dermatitis, seborrhaeic dermatitis, dermatitis herpetiformis, eczema, erythema, granuloma annulare, and inflammation caused by nettles, insect bites, poison ivy and/or poison oak, psoriasis and psoriatic arthritis and ophthalmic disorders including conjunctivitis, allergic conjunctivitis, dry eye disease, uveitis, blepharitis, meibomianitis, keratitis, conjunctival hyperaemia, Graves' ophthalmopathy, and Sjogren's syndrome.

Preferred compounds of general formula (Ia) and pharmaceutically acceptable salts thereof include compounds wherein R1 is carboxamide or cyano, compounds wherein R2 is hydrogen or halogen, compounds wherein R3 is hydrogen or methyl and compounds wherein Het′ denotes a substituted or an unsubstituted pyridyl in which the substitution may ideally be a halogen particularly Cl. More particularly preferred compounds include compounds of formula (Ib):

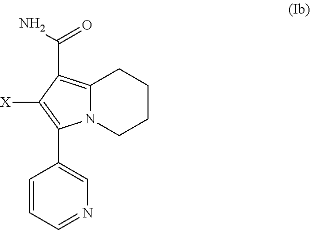

and pharmaceutically acceptable salts thereof,
in which X denotes a halogen atom, preferably a chlorine or bromine atom.

Further preferred compounds of the invention include compounds of formula (Ic), (Id) and (Ie) below:

(Ic)

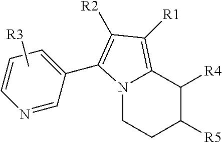

| Compound | | | | | |
|---|---|---|---|---|---|
| No | R1 | R2 | R3 | R4 | R5 |
| 1 | CONH2 | H | 4-H | H | H |
| 2 | CONH2 | Cl | 4-H | H | H |
| 3 | CN | H | 4-Me | H | H |
| 4 | CONH2 | H | 4-Me | H | H |
| 5 | CONH2 | Cl | 4-H | OH | H |
| 6 | CN | Cl | 4-H | OH | H |
| 7 | CONH2 | Cl | 4-H | OMe | H |
| 8 | CN | Cl | 4-Me | H | H |
| 9 | CONH2 | Cl | 4-Me | H | H |
| 13 | CN | H | 4H | H | H |
| 14 | CN | Cl | 4H | H | H |
| 16 | CN | Cl | 4H | OMe | H |

(Id)

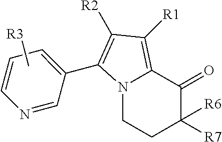

| Compound | | | | | |
|---|---|---|---|---|---|
| No | R1 | R2 | R3 | R6 | R7 |
| 10 | CN | Cl | 4-H | Cl | Cl |
| 11 | CONH2 | Cl | 4-H | H | H |
| 15 | CONH2 | Cl | 4-H | Cl | Cl |

(1e)

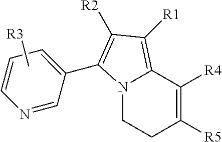

| Compound | | | | | |
|---|---|---|---|---|---|
| No | R1 | R2 | R3 | R4 | R5 |
| 12 | CN | Cl | 4-H | H | H |

Novel compounds of general formula (Ia) and pharmaceutically acceptable salts thereof also form part of the invention. Novel compounds include compounds of formula (Ia) wherein R3 is methyl in particular compounds of formula (Ic) wherein R3 is methyl, and wherein Het denotes 3-pyridyl, in particular wherein R1 is CN or CONH2 and R2 is H or Cl.

A further preferred novel compound is a compound of formula (Id) in which R6 and/or R7 are halogen in particular Cl, more particularly where in R3 is H, R2 is CONH2 and R1 is halogen more particularly Cl or Br.

Novel compounds of formula (Ia) may be selected from:
3-(4-Methyl-pyridin-3-yl)-5,6,7,8-tetrahydro-indolizine-1-carbonitrile
3-(4-Methyl-pyridin-3-yl)-5,6,7,8-tetrahydro-indolizine-1-carboxylic acid amide
2-Chloro-3-(4-Methyl-pyridin-3-yl)-5,6,7,8-tetrahydro-indolizine-1-carbonitrile
2-Chloro-3-(4-methyl-pyridin-3-yl)-5,6,7,8-tetrahydro-indolizine-1-carboxylic acid amide
2,7,7-Trichloro-8-oxo-3-pyridin-3-yl-5,6,7,8-tetrahydro-indolizine-1-carboxylic acid amide

For therapeutic administration according to the present invention, the compounds of formula (Ia) or a pharmaceutically acceptable salt thereof, may be employed in the form of its free base, but less preferably can also be used in the form of a pharmaceutically acceptable salt, e.g. the hydrochloride, mesylate, tosylate, or salts of other pharmaceutically acceptable acids.

Other salts of the compound of formula (Ia), or a pharmaceutically acceptable salt thereof with pharmaceutically acceptable acids may also be utilised in therapeutic administration, for example salts derived from the compound of formula (Ia), or a pharmaceutically acceptable salt thereof of the base with stronger acids including, but not limited to, hydrobromic acid, hydrochloric acid, phosphoric acid, methanesulphonic acid and p-toluene sulphonic acid.

All solvates and all alternative physical forms of the compound of formula (Ia) or a pharmaceutically acceptable salt thereof or its pharmaceutically acceptable derivatives as described herein, including but not limited to alternative crystalline forms, amorphous forms and polymorphs are also within the scope of this invention, and all references to the compound of formula (Ia) or a pharmaceutically acceptable salt thereof herein include all pharmaceutically acceptable salts, and all solvates and alternative physical forms thereof.

For therapeutic administration according to the invention, the compound of formula (Ia), or its pharmaceutically acceptable salts or solvates may be administered in pure form, but will preferably be formulated into any suitable pharmaceutically acceptable and effective composition which provides effective levels of the active ingredient in the body.

The treatment of dermatological and ophthalmic disorders may include administering the compound of formula (Ia), or a pharmaceutically acceptable salt thereof as 0.01 to 10% preferably 0.01-2% solutions such as eye drops, administered 1-10 times per day, or as 0.01-2% crèmes, lotions, gels, etc. to be administered 1-5 times per day.

Preferably, the compound of formula (Ia), or a pharmaceutically acceptable salt thereof is administered independently of any other medication.

The invention provides a pharmaceutical composition comprising
a) a compound of formula (Ia), or a pharmaceutically acceptable salt thereof and
b) one or more compatible and pharmaceutically acceptable excipients, diluents or adjuvants.

The invention provides a topical pharmaceutical composition comprising
a) a compound of formula (Ia), or a pharmaceutically acceptable salt thereof and
b) one or more compatible and pharmaceutically acceptable excipients, diluents or adjuvants.

The invention provides an ocular pharmaceutical composition comprising
a) a compound of formula (Ia) or a pharmaceutically acceptable salt thereof and
b) one or more compatible and pharmaceutically acceptable excipients, diluents or adjuvants.

The compositions for use in the invention may contain from 0.01-0.99% preferably 0.1% to 99% by weight, more preferably from 0.01-10% by weight, of the active material, depending on the method of administration.

In general, the medical practitioner will determine the posology which he or she considers the most appropriate as a function of the age, weight and factors pertaining to the compound and to the individual to be treated.

The choice of the most appropriate pharmaceutical compositions is within the skill of the person skilled in the art.

Suitable formulations include, but are not limited to, fluid forms, such as liquids, blends, emulsions, mixtures, solutions and suspensions in aqueous or non-aqueous vehicles; and more gelatinous, thick or viscous forms, such as balms, creams, gels, liniments, lotions, ointments, rubs and unguents, comprising emulsions, mixtures, solutions and suspensions in aqueous or non-aqueous vehicles.

In such liquid preparations, any non-aqueous vehicles may comprise oils, for example almond oil, fractionated coconut oil and olive oil; oily esters such as esters of glycerine, propylene glycol, or ethyl alcohol, e.g. ethyl oleate, and semi-synthetic glycerides; glycols, such as propylene glycols and polyethylenes glycol; and liquid paraffin.

These compositions may also contain adjuvants such as suspending agents, for example methyl cellulose, gelatin, hydroxyethylcellulose, carboxymethyl-cellulose, aluminium stearate gel, hydrogenated fats; emulsifying agents, for example lecithin, sorbitan monooleate, or acacia; preservatives, for example methyl or propyl hydroxybenzoate or sorbic acid; polysorbates, for example Tween 80 and if desired conventional colouring agents and wetting agents; and inert diluents such as sucrose, lactose or starch.

Liquid preparations are more suitable for topical ocular administration. It is preferred that the more viscous compositions are used for topical dermal administration.

The compositions may be long-acting formulations, e.g. administered by implantation (subcutaneously) with suitable polymeric or hydrophobic materials (for example as an emulsion in an acceptable oil) or ion exchange resins, or as sparingly soluble derivatives, for example, as a sparingly soluble salt.

The invention provides a process for preparing a pharmaceutical composition comprising bringing into association
a) a compound of formula (Ia), or a pharmaceutically acceptable salt thereof and
b) one or more compatible and pharmaceutically acceptable excipients, diluents or adjuvants.

The compositions may be prepared by conventional methods of blending. Repeated blending operations may be used to distribute the active agent throughout the compositions. Such operations are of course conventional in the art.

Ocular compositions should in general be sterile. The sterilisation may be carried out, for example by asepticising filtration, by incorporating sterilizing agents into the composition, by irradiation or by heating. Solutions and emulsions may be e combination of the components can be dissolved in water for injection and filter sterilised before filling into a suitable vial or ampoule.

Ocular suspensions cannot be sterilised by filtration, but can be sterilized by exposure to ethylene oxide before suspending in the sterile vehicle. Advantageously, a surfactant or wetting agent is included in the composition to facilitate uniform distribution of the active component.

The compositions for use in the invention may contain from 0.01% to 99% by weight, more preferably from 0.1% to 99% more preferably from 0.01-10% by weight, of the active material, depending on the method of administration.

Compounds of the invention may be prepared, in known manner, in a variety of ways

Compounds of formula (III) below, where R2=chlorine or bromine and R4 and R5 are both hydrogen can be prepared by hydrolysing a compound of formula (IV) for example in the presence of a strong base such as sodium hydroxide in the presence of hydrogen peroxide.

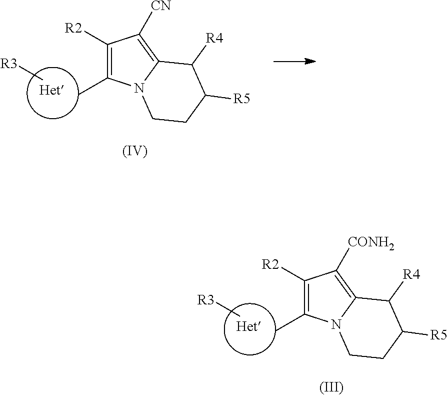

Compounds of formula (IV) below, where R2=chlorine or bromine and R4 and R5 are both hydrogen can be prepared by reducing a compound of formula (V) for example using hydrogen gas in the presence of a suitable catalyst such as palladium on charcoal.

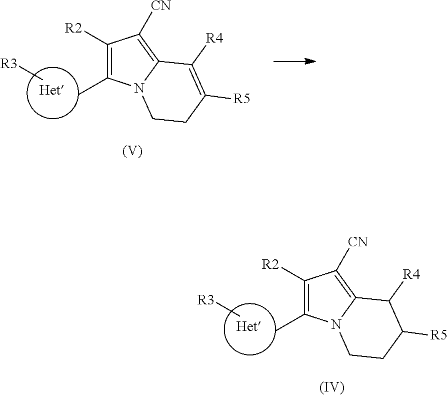

Compounds of formula (V), where R2=chlorine or bromine and R4 and R5 is hydrogen, can in turn be made by introducing a leaving group onto the hydroxyl group in the 8-position and subjecting this to a beta-elimination reaction under basic conditions. For instance by reacting a compound of formula (VI) with methane sulphonyl chloride in the presence of a base such as triethylamine to give the mesylate ester and then subjecting this to elimination in the presence of a strong base such as DBU.

Alternatively, compounds of formula (V), where R2=chlorine or bromine, R4 and R5 is hydrogen, can in turn be made by treating a compound of formula (VI) under acidic conditions by the use of, for example, p-toluenesulphonic acid to cause the elimination of water.

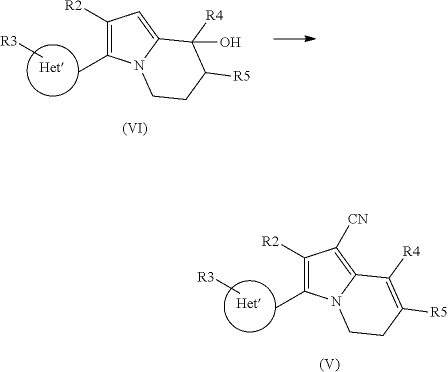

Compounds of formula (VI) where R2=chlorine or bromine and R4 and R5 is hydrogen, can in turn be made by treating a compound of formula (VII) with Hydrogen gas in the presence of a catalyst such as palladium on charcoal to effect the reduction of the 8-keto-group and concomitant hydrogenolysis of the 7,7-dichloro-group.

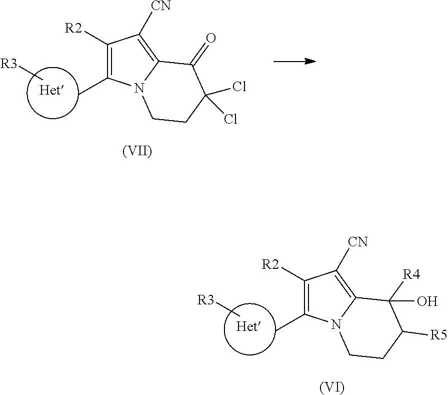

Compounds of formula (VII) below, where R2=chlorine can be made by treating a compound of formula (VIII) (R2=H) with an excess of a chlorinating agent such as N-chlorosuccinimide in a suitable solvent such as acetonitrile. Compounds of formula (VII) below, where R2=bromine can be made by treating a compound of formula (VIII) with an excess of a brominating agent such as N-bromosuccinimide in a suitable solvent such as acetonitrile.

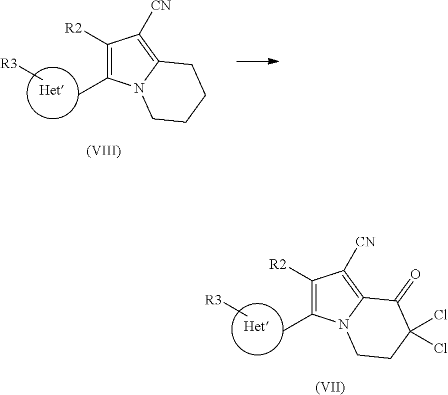

Compounds of formula (VIII) below where R2=chlorine can be made by treating an ester of formula (IX) with 2,3-dichloroacrylonitrile in the presence of p-toluene sulphonyl chloride and a base such as triethylamine

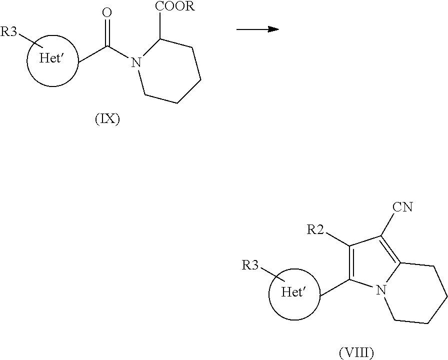

Compounds of formula (X) below, where R2, R4 and R5 are all hydrogen can be prepared by hydrolysing a compound of formula (XI) for example in the presence of a strong base such as sodium hydroxide in the presence of hydrogen peroxide

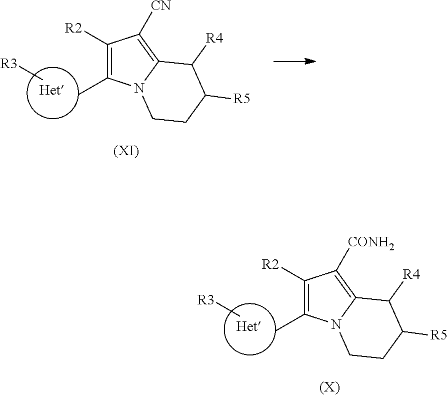

Compounds of formula (XI) below, where R2, R4 and R5 are all hydrogen can be prepared by treating an ester of formula (XII) with a base such as sodium hydroxide followed by reaction with 2-chloroacrylonitrile in the presence of p-toluene sulphonyl chloride and a base such as triethylamine.

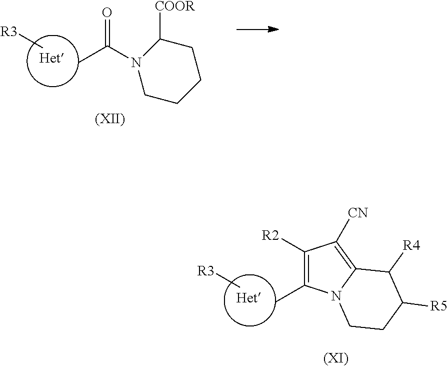

Other methods will be apparent to the chemist skilled in the art as will be the methods for preparing the corresponding starting materials and intermediates.

The anti-inflammatory activity of the present compounds of the invention can be demonstrated by various assays known in the art, for example for their ability to inhibit IL-1β stimulated IL-8 secretion in human umbilical vein endothelial cells (HUVECs) using Sandwich Enzyme-Linked Immunosorbent Assay (ELISA).

The activity of selected compound(s) on a wider range of pro-inflammatory Cytokines such as GM-CSF, MCP-1, siCAM-1, IL-6 can be used to determine the effect of IL-1β and selected compound(s) on these cytokine/chemokine secretions in human umbilical vein endothelial cells (HUVECs).

The activity of selected compound(s) was determined in an In vivo Antiinflammatory Assay Effect by Topical Application in a Mouse Allergic Contact Dermatitis Model

### 1. In Vitro Anti-Inflammatory Assay—IL-18 Stimulated IL-8 Secretion Assay in HUVEC Cells

Anti-inflammatory screening was established in human umbilical vein endothelial cells (HUVECs) to screen the test compounds for their ability to inhibit IL-1β stimulated IL-8 secretion from these cells. The level of secreted IL-8 in the HUVEC media was detected using a Quantikine™ Sandwich Enzyme-Linked Immunosorbent Assay (ELISA) from R&D Systems.

Cells and media were obtained from Cascade Biologics™ via Invitrogen. HUVECs were seeded in sterile 96-well tissue culture treated plates (0.1 ml per well, containing 2,400 cells; 8000 cells/cm²). The seeded plates were incubated in a humidified incubator at 37 C/5% CO₂. After 24 h, the media was replaced with fresh media (80 μl). The test compounds were serially diluted from 10 mM DMSO stocks with media to obtain dilutions 10× the final concentration. The compounds (10 μl) were added to the plates 30 min prior to the addition of 10 μl of 5 ng/ml stimulant IL-1β (final concentration 0.5 ng/ml). After incubating for 24 h, the plates were centrifuged at 200×g for 5 min (room temperature) and the media from each well transferred to a 96-well storage plate. Basal samples were assayed neat, but media from IL-1β stimulated cells was diluted in the calibrator diluent accompanying the IL-8 ELISA kit. The ELISA was performed according to the manufacturer's instructions.

### Results

A dose of 0.5 ng/ml IL-1β was chosen as a submaximal dose to observe the inhibition of IL-8 secretion with the test compounds.

Test compounds were initially screened at 10⁻⁵M and 10⁻⁶M for their ability to inhibit IL-1β stimulated IL-8 secretion (Table 1).

TABLE 1

| Compound | IC50 | % Inhib at | % Inhib at |
|---|---|---|---|
| Number | (μM) | 10 μM | 1 μM |
| 1 | | 34.7 | 31.8 |
| 2 | 0.35 | 59.5 | 49.4 |
| 3 | | 35 | 34.4 |
| 4 | | 30.8 | 15.9 |
| 5 | 0.76 | 52.4 | 29 |
| 6 | | 21.8 | 52.2 |
| 7 | | 32.1 | 1.4 |
| 8 | | 39.6 | 5.5 |
| 9 | 0.51 | 49.6 | 42.2 |
| 10 | | 35.7 | 18.8 |
| 11 | | 31.7 | 32.9 |
| 12 | 1.6 | 52.7 | 22.7 |

### 2. In Vitro Anti-Inflammatory Assay—Effect of Compound (2) on Variety of IL-1β Stimulated Cytokines/Chemokines in HUVECs

A cytokine protein array was performed to determine the effect of IL-1β and compound (2) on other cytokine/chemokine secretions in human umbilical vein endothelial cells (HUVECs). From the results, five additional cytokines (GM-CSF, MCP-1, siCAM-1, IL-6) were chosen to investigate further using individual ELISAs and a full dose response curve for compound (2).

### Method

### Human Cytokine Protein Array

The HUVEC IL1β secretion assay was performed using a 24-well plate format similar to that described for IL-8 secretion above (0.5 ml per well containing 16,000 cells; 8000 cells/cm²). Media was collected from untreated cells (basal) and cells treated with either 0.5 ng/ml IL-1β only, 10⁻⁵M compound (2) only or IL-1β stimulated plus compound (2). Media from 2 wells were pooled for each condition.

The Proteome Profiler™ human cytokine array panel A array kit (R&D Systems) consists of selected capture antibodies against 36 different cytokines/chemokines spotted on nitrocellulose membranes. The array method used was that detailed in the manufacturer's instructions. The HUVEC supernates were preincubated with the cocktail of biotinylated detection antibodies, then the mixture was added to the membranes (one for each condition). After washing, the membranes were incubated with streptavidin-horseradish peroxidase and chemiluminescent detection reagents sequentially. The light signal generated was quantified using a BioRad XRS chemiluminescence detector. The results were expressed as a percentage of the positive control on each membrane.

### Measurement of Cytokine Secretion

Using the results of the protein array, the effect of compound (2) on the secretion of five other cytokines stimulated by IL-1β was also measured in HUVECs. These included: GROα, MCP-1, GM-CSF, sICAM-1 and IL-6. The HUVECs were seeded in sterile 24-well tissue culture treated plates (as described above). The cells were treated with increasing doses of compound (2) prior to stimulating with IL-1β as described above. All cytokines were measured using the appropriate Quantikine® Sandwich ELISA from R&D Systems according to the manufacturer's instructions.

### Effect of Compound (2) on IL-1β Stimulated Cytokine Secretion in HUVECs

Confirming the results of the protein assay, IL-1β simulated secretion of GROα, IL-8, MCP-1, IL-6, and GM-CSF in HUVECs compared to untreated basal cells. Soluble ICAM-1 was not detected in the IL-1β stimulated HUVEC media. Increasing concentration of compounds (2) had little effect on IL-1β stimulated MCP-1 and GROα levels were reduced by only ˜25%. Compound (2) dose dependently decreased GM-CSF levels similar to that observed with IL-8. Compound (2) increased IL-6 levels further rather than decreased them.

The cytokine/chemokine profile observed was characteristic of that predicted for IL-1β stimulation of endothelial cells. Of the cytokines/chemokines tested further, compound (2) inhibited IL-8 and GM-CSF to a similar extent.

### 3. In Vivo Anti-Inflammatory Assay—Effect of Topical Application of Compound (2) in the Mouse Allergic Contact Dermatitis Model

### Method

BALB/c derived male mice, weighing 23±2 g were housed in individually ventilated cages racks (IVC Racks, 36 Mini Isolator systems) throughout the experiment. Five mice per group were kept in a cage (in cm, 26.7 length×20.7 width×14.0 height) and maintained under controlled temperature (21-23° C.) and humidity (50%-70%) with 12-hour light/dark cycles. The animals were given free access to sterilized lab chow and reverse osmosis (RO) water ad libitum. All aspects of this work, i.e. housing, experimentation and disposal of animals, are performed in general accordance with the Guide for the Care and Use of Laboratory Animals (National Academy Press, Washington, D.C., 1996).

Compound (2) was prepared as 150, 50 and 15 mg/ml in vehicle, acetone:ethanol/1:1. The preshaven abdomens of five groups of 5 BALB/c test mice were sensitized by application of 100 μL of 1.5% oxazolone solution dissolved in acetone. Seven days after the initial sensitization, test substance (0.3, 1 and 3 mg in 20 μl/ear) and vehicle (acetone:ethanol/1:1, 20 μL/ear) were each administered topically to the anterior and posterior surfaces of the right ear 30 minutes before and 15 minutes after challenge by the second application of oxazolone (1% in acetone, 20 μL/ear). As a positive control, dexamethasone (0.1 mg/ear) was administered topically using the same treatment regime as the test compound. Twenty-four hours after the second application of oxazolone, the ear thickness of each mouse was measured with a Dyer model micrometer gauge. Ear edema was calculated by subtracting the thickness of the left ear (normal control) from the right ear (treated ear). Percent inhibition was calculated according to the formula: [(I_{c}−Iₜ)/I_{c}]×100%, where I_{c} and Iₜ refer to increase of ear thickness (mm) in control and treated mice, respectively. A 30 percent or more (30%) inhibition in ear swelling relative to the vehicle control is considered significant and indicates possible anti-inflammatory activity.

### Results

Compound (2) at 0.3, 1 and 3 mg/ear×2 caused dose-dependent, moderate to significant inhibition (25%, 29% and 40%, respectively, relative to the vehicle control (acetone:ethanol/1:1, 20 μL/ear). The dexamethasone control gave 72% inhibition.

Topical application of compound (2) dose dependently inhibited inflammation in the mouse allergic contact dermatitis model induced by oxazolone. Although, compound 2 was not as potent as dexamethasone, it should be noted that the formulation of compound (2) was not completely soluble; hence this may be an underestimate of activity.

The following Examples serve only to illustrate the invention and it's practice. The examples are not to be construed as limitations on the scope or spirit of the invention.

Structures of isolated novel compounds were characterised and confirmed by ¹H NMR, LCMS and/or other appropriate analyses.

### Preparation of Starting Materials

### Ethyl-1-(Pyridine-3-carbonyl)-piperidine-2-carboxylate

To a stirred solution of ethyl pipecolinate (13.6 g, 76 mmol) in triethylamine (18 ml) and dichloromethane (600 ml) at 50° C. was added nicotinyl chloride hydrochloride (15 g), portionwise, over 1 hour, as a solid. The solution was stirred at 50° C. for 5 hours, then washed with 2M sodium hydroxide solution (2×400 ml) and then with saturated brine (1×300 ml), then dried over MgSO₄, filtered and evaporated under reduced pressure to give a pure yellow oil (19.18 g, 73.1 mmol). This oil was used without further purification; ¹H NMR (300 MHz, CDCl₃): δ_{H}=0.9 (3H, t) 1.5-1.7 (4H, m), 2.3 (2H, m), 3.2-3.5 (2H, m), 4.2 (2H, m), 5.4 (1H, m), 7.3 (1H, m), 7.7 (1H, m) 8.7 (2H, m).

### 1-(pyridine-3-carbonyl)-piperidine-2-carboxylate sodium salt

To a solution of Ethyl-1-(Pyridine-3-carbonyl)piperidine-2-carboxylate in acetonitrile (250 ml) and ethanol (22 ml) was added sodium hydroxide (3 g, 75 mmol) in water (2 ml) dropwise. After a few minutes, a white solid precipitated. The reaction mixture was left to stir for 18 h at room temperature. The white suspension was then filtered and washed with acetonitrile. The precipitate was dried in a vacuum oven at 50° C. for 4 hours to give the sodium salt as a white solid (16.8 g, 97%).

### 1-(4-Methyl-pyridine-3-carbonyl)-piperidine-2-carboxylic acid ethyl ester

To a stirred solution of 4-methyl-pyridine-2-carboxylic acid (1 g) in dichloromethane (20 ml) was added ethyl pipecolinate (1.25 ml), HBTU (1.15 g), and 4-methylmorpholine (1.2 ml) and the reaction stirred at room temperature for 18 h. The reaction mixture was washed with water (3×50 ml), brine (1×20 ml), then dried (MgSO₄), filtered and evaporated under reduced pressure to yield a pale yellow oil (1.8 g, 88%). This oil was used without further purification; ¹H NMR (300 MHz, CDCl₃): δ_{H}=1.0 (3H, t), 1.5-1.7 (4H, m), 2.2 (3H, s), 2.3 (2H, m), 3.0-3.4 (2H, m), 4.2 (2H, m), 5.4 (1H, m), 7.1 (1H, m), 8.3 (1H, m) 8.4 (2H, m).

### 1-(4-methyl-pyridine-3-carbonyl)-piperidine-2-carboxylate sodium salt

1-(4-Methyl-pyridine-3-carbonyl)-piperidine-2-carboxylic acid ethyl ester (1.28 g) was reacted under the conditions described for the corresponding ethyl-1-(pyridine-3-carbonyl)-piperidine-2-carboxylate to yield the desired product as a white solid that was used without further purification (0.86 g, 69%).

### EXAMPLE 1

### 3-pyridin-3-yl-5,6,7,8-tetrahydro-indolizine-1-carboxamide (Compound 1)

3-pyridin-3-yl-5,6,7,8-tetrahydro-indolizine-1-carbonitrile (0.3 g, 1.3 mmol) was dissolved in methanol (25 ml) containing a 35% aqueous solution of hydrogen peroxide (4 ml) and sodium hydroxide (0.43 g, 13 mmol) dissolved in water (1 ml). The reaction was stirred at room temperature for 18 h. The solvent was evaporated under reduced pressure and the resulting crude product redissolved in dichloromethane (10 ml), and the organic phase washed with water (2×10 ml), brine (1×10 ml) then dried (MgSO₄), filtered and evaporated under vacuum to give the product as a yellow solid (0.20 g). The crude product was purified by silica chromatography using 10% methanol in dichloromethane as the eluant, to give the target compound a white solid (0.12 g, 40%).

¹H NMR (300 MHz, CDCl₃): δ_{H}=1.98 (4H, m), 3.25 (2H, m), 3.95 (2H, m), 5.5 (1H, bs), 6.5 (1H, s), 7.37 (1H, m), 7.75 (1H, m), 8.6 (1H, m). 8.7 (1H, m).

### EXAMPLE 2

### 2-Chloro-3-pyridin-3-yl-5,6,7,8-tetrahydro-indolizine-1-carboxamide (Compound 2)

2-Chloro-3-pyridin-3-yl-5,6,7,8-tetrahydro-indolizine-1-carbonitrile (1 g, 3.89 mmol) was dissolved in methanol (60 ml) containing a 35% aqueous solution of hydrogen peroxide (43 ml) and sodium hydroxide (1.55 g, 38.8 mmol) dissolved in water (5 ml). The reaction was stirred at room temperature for 18 h The solvent was evaporated under reduced pressure and the resulting crude product dissolved in dichloromethane (50 ml), and the organic phase washed with water (2×50 ml), brine (1×50 ml) then dried (MgSO₄), filtered and evaporated under vacuum to give the product as a yellow solid (0.20 g). The crude product was purified by silica chromatography using 10% methanol in dichloromethane as the eluant to give the title compound a white solid (0.70 mg, 65%). ¹H NMR (300 MHz, CDCl₃): δ_{H}=1.98 (4H, m), 3.2 (2H, m), 3.8 (2H, m), 5.3 (1H, bs), 6.7 (1H, bs), 7.41 (1H, m), 7.8 (1H, m), 8.7 (2H, m).

### EXAMPLE 3

### 3-(4-Methyl-pyridin-3-yl)-5,6,7,8-tetrahydro-indolizine-1-carbonitrile (Compound 3)

A suspension of p-toluenesulfonyl chloride (0.72 g), sodium 1-(4-methyl-pyridine-3-carbonyl)-piperidine-2-carboxylate (0.55 g) and 2-chloroacrylonitrile (0.52 g) in dichloroethane (25 ml) was stirred for 20 minutes then triethylamine (1 ml) was then added dropwise. The resulting solution was heated at 50° C. for 4 hours. The reaction mixture was cooled, washed with water (4×25 mL) and brine (1×10 ml), then dried over MgSO₄, filtered and evaporated under reduced pressure to give the product as a crude brown oil (0.7 g, 96.5%). The crude product was purified by silica chromatography using 10% methanol in dichloromethane as the eluant, followed by trituration in diethylether to yield the title compound as a beige solid (0.48 g, 66%); ¹H NMR (300 MHz, CDCl₃): δ_{H}=1.98 (4H, m), 2.4 (3H, s), 3.1 (2H, m), 3.6 (2H, m), 6.5 (1H, s), 7.8 (1H, m), 8.7 (2H, m).

### EXAMPLE 4

### 3-(4-Methyl-pyridin-3-yl)-5,6,7,8-tetrahydro-indolizine-1-carboxylic acid amide (Compound 4)

3-(4-Methyl-pyridin-3-yl)-5,6,7,8-tetrahydro-indolizine-1-carbonitrile (0.02 g) was reacted under the conditions described for example 2 to yield the title compound as a pale yellow solid (0.01 g, 60%); ¹H NMR (300 MHz, CDCl₃): δ_{H}=1.98 (4H, m), 2.4 (3H, s), 3.2 (2H, m), 3.7, 5.5 (bs), 6.5 (1H, s), 6.7 (bs), 7.9 (1H, m), 8.7 (2H, m).

### EXAMPLE 5

### 2-Chloro-8-hydroxy-3-pyridin-3-yl-5,6,7,8-tetrahydro-indolizine-1-carboxylic acid amide (Compound 5)

2-Chloro-8-hydroxy-3-pyridin-3-yl-5,6,7,8-tetrahydro-indolizine-1-carbonitrile (1 g) was reacted under the conditions used for example 2 to yield the title compound as a beige solid (0.68 mg, 67%); ¹H NMR (300 MHz, CDCl₃): δ_{H}=2.1 (4H, m), 3.7-3.9 (2H, m), 5.2 (1H, m), 5.9 (1H, bs), 6.1 (1H, s), (6.9, bs), 7.4 (1H, m), 7.7 (1H, m), 8.7 (2H, m).

### EXAMPLE 6

### 2-Chloro-8-hydroxy-3-pyridin-3-yl-5,6,7,8-tetrahydro-indolizine-1-carbonitrile (Compound 6)

2-Chloro-8-oxo-3-pyridin-3-yl-5,6,7,8-tetrahydro-indolizine-1-carbonitrile (0.50 g, 0.18 mmol) was dissolved in methanol (6 ml) and sodium borohydride (0.21 g, 0.54 mmol) added portionwise over 10 minutes. The reaction mixture was stirred at room temperature for 1 hour, then quenched with water (10 ml) and extracted with ethyl acetate (3×10 ml). The combined organic fractions were dried (MgSO₄), filtered and evaporated under vacuum to give a brown oil (0.35 g, 70%). The crude oil was purified by silica chromatography using 10% methanol in dichloromethane as the eluant to yield the title compound as a pale brown solid (0.29 g, 58%); ¹H NMR (300 MHz, CDCl₃): δ_{H}=2.1 (4H, m), 3.7-3.9 (2H, m), 5.25 (1H, m), 7.5 (1H, m), 7.75 (1H, m), 8.75 (2H, m).

### EXAMPLE 7

### 2-Chloro-8-methoxy-3-pyridin-3-yl-5,6,7,8-tetrahydro-indolizine-1-carboxylic acid amide (Compound 7)

2-Chloro-8-methoxy-3-pyridin-3-yl-5,6,7,8-tetrahydro-indolizine-1-carbonitrile (0.035 g) was reacted under the conditions described for example 2 to yield the title compound as a pale brown solid (0.025 g, 71%); ¹H NMR (300 MHz, CDCl₃): δ_{H}=2.1 (4H, m), 3.3 (3H, m), 3.5 (2H, s), 3.6-3.8 (2H, m), 5.2 (1H, m), 5.6 (1H, bs), 6.9 (1H, bs), 7.4 (1H, m), 7.8 (1H, m), 8.7 (2H, m).

### EXAMPLE 8

### 2-Chloro-3-(4-Methyl-pyridin-3-yl)-5,6,7,8-tetrahydro-indolizine-1-carbonitrile (Compound 8)

To a solution of p-toluenesulfonyl chloride (1.1 g) and 2,3-dichloroacrylonitrile (0.82 ml) in 1,2-dichloroethane (26 ml), was added sodium 1-(4-methyl-pyridine-3-carbonyl)-piperidine-2-carboxylate (1.5 g) and the resulting suspension stirred at room temperature for 1 hour. After this time triethylamine (0.82 ml) was added dropwise to the solution and the reaction mixture was heated at 40° C. for 24 hours. The reaction mixture was cooled, washed with water (4×250 ml) and brine (1×100 ml), then dried over MgSO₄, filtered and evaporated under reduced pressure to give the product as a brown oil (1.1 g, 98%). The oil was triturated with diethylether (20 ml) to give the title compound as a beige solid that required no further purification (0.8 g, 80%); ¹H NMR (300 MHz, CDCl₃): δ_{H}=1.98 (4H, m), 2.3 (3H, s), 3.0 (2H, m), 3.4-3.7 (2H, m), 8.4 (1H, m), 8.6 (2H, m).

### EXAMPLE 9

### 2-Chloro-3-(4-methyl-pyridin-3-yl)-5,6,7,8-tetrahydro-indolizine-1-carboxylic acid amide (Compound 9)

3-(4-Methyl-pyridin-3-yl)-5,6,7,8-tetrahydro-indolizine-1-carbonitrile (0.12 g) was reacted under the conditions described for example 2 to give the title compound as a pale yellow solid that required no further purification; ¹H NMR (300 MHz, CDCl₃): δ_{H}=1.9 (4H, m), 3.3 (2H, m), 3.5-3.7 (2H, m), 5.3 (1H, bs), 6.7 (1H, bs), 8.4 (1H, m), 8.6 (2H, m).

### EXAMPLE 10

### 2,7,7-Trichloro-8-oxo-3-pyridin-3-yl-5,6,7,8-tetrahydro-indolizine-1-carbonitrile (Compound 10)

3-Pyridin-3-yl-5,6,7,8-tetrahydro-indolizine-1-carbonitrile (17.19 g, 77 mmol) and N-chlorosuccinimide (53.76 g, 380 mmol) were combined in acetonitrile (600 ml), and heated under reflux at 85° C. for 4 h to give a red solution. The solvent was evaporated under reduced pressure. Water (600 mL) was added and the solution adjusted to pH 8 by addition of sodium bicarbonate. The resulting orange solid was filtered, washed with water (2×200 mL) and dried in a vacuum oven at 50° C. for 1 h affording the product as a brown solid (20.34 g, 77.8%). The product was not purified further; ¹H NMR (300 MHz, CDCl₃): δ_{H}=3.2 (2H, m), 4.1 (1H, m), 4.3 (1H, m), 7.6 (1H, m), 7.9 (1H, m), 8.8 (2H, m).

### EXAMPLE 11

### 2-Chloro-8-oxo-3-pyridin-3-yl-5,6,7,8-tetrahydro-indolizine-1-carboxylic acid amide (Compound 11)

2-Chloro-8-oxo-3-pyridin-3-yl-5,6,7,8-tetrahydro-indolizine-1-carbonitrile (0.02 g) was reacted under the conditions described for example 15 to yield the title compound as a yellow oil that was used without further purification, (0.005 g, 25%); ¹H NMR (300 MHz, CDCl₃): δ_{H}=2.3 (2H, m), 2.8 (2H, m), 4.1 (2H, m), 5.7 (1H, bs), 7.5 (1H, m), 7.8 (1H, m), 8.8 (2H, m).

### EXAMPLE 12

### 2-Chloro-3-pyridin-3-yl-5,6-dihydro-indolizine-1-carboxylic acid amide (compound 12)

2-Chloro-3-pyridin-3-yl-5,6-dihydro-indolizine-1-carbonitrile (0.05 g) was reacted under the conditions described for example 2 to yield the title compound as a pale yellow solid (0.01 g, 20%); ¹H NMR (300 MHz, CDCl₃): δ_{H}=2.5 (2H, m), 3.9 (2H, m), 5.9 (1H, bs), 6.1 (1H, m), (6.7 (1H, bs), 7.5-7.6 (2H, m), 7.8 (1H, m), 8.7 (2H, m).

### EXAMPLE 13

### 3-pyridin-3-yl-5,6,7,8-tetrahydro-indolizine-1-carbonitrile (Compound 13)

To a solution of p-toluenesulfonyl chloride (2.46 g, 12.9 mmol) in dichloromethane (25 ml), 2-chloroacrylonitrile (1.18 ml) was added dropwise to give a clear solution. Triethylamine (1 ml) was then added to the solution. Sodium 1-(pyridine-3-carbonyl)-piperidine-2-carboxylate (2.89 g, 12.4 mmol) was then added over 1 h under stirring, followed by further triethylamine (3 ml). The solution was heated under reflux at 40° C. for 4 hours. The reaction mixture was washed with 2N HCl (4×250 ml) and brine (1×100 ml), then dried over MgSO₄, filtered and evaporated under reduced pressure to give the product as a brown oil (2.67 g, 96.5%). Analysis revealed presence of 2 regioisomers in a ratio of (5:1), so the crude oil was purified by silica chromatography using 20% ethyl acetate in hexane as the eluant. The major isomer was isolated as a pale yellow oil (1.93 g, 70%); ¹H NMR (300 MHz, CDCl₃): δ_{H}=1.9-2.1 (4H, m), 3.1 (2H, m), 3.9 (2H, m), 6.5 (1H, s), 7.4 (1H, m), 7.7 (1H, m), 8.7 (2H, m).

### EXAMPLE 14

### 2-Chloro-3-pyridin-3-yl-5,6,7,8-tetrahydro-indolizine-1-carbonitrile (Compound 14)

To a solution of p-toluenesulfonyl chloride (1.95 g, 10.2 mmol) and 2,3-dichloroacrylonitrile (1.95 ml) in dichloromethane (75 ml), was added sodium 1-(pyridine-3-carbonyl)-piperidine-2-carboxylate (1.5 g) and the resulting suspension stirred at room temperature for 1 hour. After this time triethylamine (1.95 ml) was added dropwise to the solution then the reaction mixture was heated under reflux at 40° C. for 4 hours. The reaction mixture was cooled, washed with 2N HCl (4×250 ml) and brine (1×100 ml), then dried over MgSO₄, filtered and evaporated under reduced pressure to give the product as a brown oil (1.5 g, 98%). The crude oil was purified by silica chromatography using 10% methanol in dichloromethane as the eluant. Product was isolated as a pale brown solid (1.03 g, 72%); ¹H NMR (300 MHz, CDCl₃): δ_{H}=2.0 (4H, m), 2.9 (2H, m), 3.8 (2H, m), 7.4 (1H, m), 7.8 (1H, m), 8.7 (2H, m).

### EXAMPLE 15

### 2,7,7-Trichloro-8-oxo-3-pyridin-3-yl-5,6,7,8-tetrahydro-indolizine-1-carboxylic acid amide (Compound 15)

2,7,7-Trichloro-8-oxo-3-pyridin-3-yl-5,6,7,8-tetrahydro-indolizine-1-carbonitrile (0.02 g) was added to conc. sulphuric acid (1 ml) and the reaction mixture stirred at room temperature for 18 h. The reaction mixture was basified to pH7 by addition of sodium bicarbonate solution, then extracted with dichloromethane (3×5 ml). The combined organic phases were washed with water (3×5 ml), brine (1×5 ml), then dried (MgSO4), filtered and evaporated under vacuum to yield the title compound as a pale yellow oil (0.002 g, 10%); ¹H NMR (300 MHz, CDCl₃): δ_{H}=3.3 (2H, m), 4.3 (2H, m), 5.8 (1H, bs), 7.6 (1H, m), 7.9 (1H, m), 8.7 (2H, m).

### EXAMPLE 16

### 2-Chloro-8-methoxy-3-pyridin-3-yl-5,6,7,8-tetrahydro-indolizine-1-carbonitrile (Compound 16)

To a stirred suspension of sodium hydride (60% dispersion in mineral oil, 4 mg) in THF (5 ml) under a nitrogen atmosphere was added 2-Chloro-8-hydroxy-3-pyridin-3-yl-5,6,7,8-tetrahydro-indolizine-1-carbonitrile (0.03 g) and the reaction mixture stirred for 1 hour, Methyl iodide (0.017 mg) was added dropwise and the reaction mixture stirred for a further 18 h at room temperature. The reaction mixture was diluted with dichloromethane (15 ml), and washed with water (3×10 ml), brine (1×10 ml) then dried (MgSO₄), filtered and evaporated under reduced pressure to yield a yellow oil that was triturated in ether (10 ml) and used without further purification (0.015 g, 49%). ¹H NMR (300 MHz, CDCl₃): δ_{H}=2.1-2.3 (4H, m), 3.55 (3H, s), 3.7-3.9 (2H, m), 5.15 (1H, m), 7.4 (1H, m), 7.75 (1H, m), 8.7 (2H, m).

1. A method of topical treatment or prophylaxis of dermatological or ophthalmic disorders in mammals by administration of a compound of formula (Ia) or a pharmaceutically acceptable salt thereof: 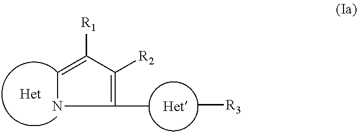
in which Het denotes a heterocyclic or carbocyclic ring condensed with a pyrrole such that it forms a substituted or unsubstituted indolizine, pyrrolothiazole, 5,6,7,8-tetrahydroindolizine, 5,6,-dihydroindolizine, dihydropyrrolothiazine or dihydropyrrolizine ring,
R1 is a carboxamide, cyano, carboxyl, alkoxy carbonyl, acyl
R2 is a hydrogen or halogen atom or an alkyl, alkenyl, trihalomethyl or cyano radical,
R3 is a hydrogen or halogen atom or a hydroxyl or alkyl radical, and
Het′ is a pyridyl, pyridyl N-oxide or thiazolyl radical,
wherein the alkyl or acyl radicals are straight or branched and contain 1 to 4 carbon atoms and the alkenyl radicals are straight or branched and contain 2 to 4 carbon atoms, and wherein, when R2 and/or R3 are halogen, they are chosen from chlorine, bromine, fluorine or iodine.

2. (canceled)

3. (canceled)

4. (canceled)

5. The method according to claim 1, wherein the dermatological disorder is selected from the group including atopic dermatitis, contact dermatitis, seborrhaeic dermatitis, dermatitis herpetiformis, eczema, erythema, granuloma annulare, and inflammation caused by nettles, insect bites, poison ivy and/or poison oak, psoriasis and psoriatic arthritis.

6. The method according to claim 1, wherein the ophthalmic disorder is selected from conjunctivitis, allergic conjunctivitis, dry eye disease, uveitis, blepharitis, meibomianitis, keratitis, conjunctival hyperaemia, Graves' ophthalmopathy, and Sjogren's syndrome.

7. A method of topical treatment or prophylaxis of rheumatoid arthritis or osteoarthritis in mammals by administration of a compound of formula (Ia) or a pharmaceutically acceptable salt thereof: 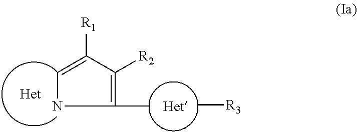
in which Het denotes a heterocyclic or carbocyclic ring condensed with a pyrrole such that it forms a substituted or unsubstituted indolizine, pyrrolothiazole, 5,6,7,8-tetrahydroindolizine, 5,6,-dihydroindolizine, dihydropyrrolothiazine or dihydropyrrolizine ring,
R1 is a carboxamide, cyano, carboxyl, alkoxy carbonyl, acyl
R2 is a hydrogen or halogen atom or an alkyl, alkenyl, trihalomethyl or cyano radical,
R3 is a hydrogen or halogen atom or a hydroxyl or alkyl radical, and
Het′ is a pyridyl, pyridyl N-oxide or thiazolyl radical,
wherein the alkyl or acyl radicals are straight or branched and contain 1 to 4 carbon atoms and the alkenyl radicals are straight or branched and contain 2 to 4 carbon atoms, and wherein, when R2 and/or R3 are halogen, they are chosen from chlorine, bromine, fluorine or iodine.

8. The method according to claim 1, wherein the compound of formula (Ia) or a pharmaceutically acceptable salt thereof is a compound in which R1 is carboxamide or cyano.

9. The method according to claim 1, wherein the compound of formula (Ia) or a pharmaceutically acceptable salt thereof is a compound in which R2 is hydrogen or halogen.

10. The method according to claim 1, wherein the compound of formula (Ia) or a pharmaceutically acceptable salt thereof is a compound in which R3 is hydrogen or methyl.

11. The method according to claim 1, wherein the compound of formula (Ia) or a pharmaceutically acceptable salt thereof is a compound in which Het denotes a substituted or an unsubstituted pyridyl.

12. The method according to claim 1, wherein the compound is of formula (Ib) or a pharmaceutically acceptable salt thereof 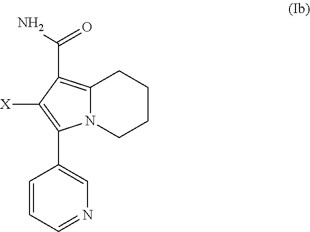
in which X denotes a halogen atom.

13. A compound of formula (Ia) or a pharmaceutically acceptable salt thereof: 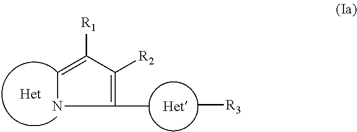
in which Het denotes a heterocyclic or carbocyclic ring condensed with a pyrrole such that it forms a substituted or unsubstituted indolizine, pyrrolothiazole, 5,6,7,8-tetrahydroindolizine, 5,6,-dihydroindolizine, dihydropyrrolothiazine or dihydropyrrolizine ring,
R1 is a carboxamide, cyano, carboxyl, alkoxy carbonyl, acyl
R2 is a hydrogen or halogen atom or an alkyl, alkenyl, trihalomethyl or cyano radical,
R3 is methyl
Het′ is a pyridyl, pyridyl N-oxide or thiazolyl radical,
wherein the alkyl or acyl radicals are straight or branched and contain 1 to 4 carbon atoms and the alkenyl radicals are straight or branched and contain 2 to 4 carbon atoms, and wherein, when R2 and/or R3 are halogen, they are chosen from chlorine, bromine, fluorine or iodine.

14. The compound according to claim 13 wherein Het denotes 3-pyridyl.

15. The compound according to claim 14 wherein R1 is CN or CONH2 and R2 is H or Cl.

16. The compound of formula (1a) of claim 13 selected from:
3-(4-Methyl-pyridin-3-yl)-5,6,7,8-tetrahydro-indolizine-1-carbonitrile;
3-(4-Methyl-pyridin-3-yl)-5,6,7,8-tetrahydro-indolizine-1-carboxylic acid amide;
2-Chloro-3-(4-Methyl-pyridin-3-yl)-5,6,7,8-tetrahydro-indolizine-1-carbonitrile;
2-Chloro-3-(4-methyl-pyridin-3-yl)-5,6,7,8-tetrahydro-indolizine-1-carboxylic acid amide; and
2,7,7-Trichloro-8-oxo-3-pyridin-3-yl-5,6,7,8-tetrahydro-indolizine-1-carboxylic acid amide.

17. A pharmaceutical composition comprising a) the compound of formula (Ia) or a pharmaceutically acceptable salt thereof of claim 13 and b) one or more compatible and pharmaceutically acceptable excipients, diluents or adjuvants.

18. The pharmaceutical composition according to claim 17 which is a topical pharmaceutical composition.

19. The pharmaceutical composition according to claim 18 which is an ocular pharmaceutical composition.
